(19) United States
(12) Patent Application Publication
(10) Pub. No.: US 2025/0197252 A1
(43) Pub. Date: Jun. 19, 2025

(54) CAP UNIT, STORAGE TANK, AND FLOWING WATER STERILIZATION DEVICE

(71) Applicant: Stanley Electric Co., Ltd., Tokyo (JP)

(72) Inventors: Masakazu INOUE, Tokyo (JP); Kazuaki KAWASAKI, Tokyo (JP); Hideaki TANAKA, Tokyo (JP); Hiroyuki KATO, Tokyo (JP); Kazuhisa SHINNO, Tokyo (JP)

(21) Appl. No.: 18/843,063

(22) PCT Filed: Feb. 27, 2023
(86) PCT No.: PCT/JP2023/007054

§ 371 Date: Aug. 30, 2024

(30) Foreign Application Priority Data
Mar. 1, 2022 (JP) 2022-031059

(51) Int. Cl. C02F 1/32; A61L 2/10

(52) U.S. Cl. CPC C02F 1/325; A61L 2/10; A61L 2202/11; A61L 2202/121; A61L 2202/122; C02F 2201/004; C02F 2201/3222; C02F 2201/3226; C02F 2201/3228; C02F 2303/04

(57) ABSTRACT

An object is to provide a cap unit capable of enhancing the sterilizing ability with respect to an object to be sterilized while maintaining water stopping properties in a housing, and capable of irradiating a desired range with ultraviolet light, as well as a storage tank and a flowing water sterilization device.

The cap unit includes: a light source unit including a light emitting element and a substrate on which the light emitting element is mounted; a housing that has an accommodation space for the light source unit and an opening at a predetermined position on a side that faces an object to be sterilized, the housing being configured to accommodate the light source unit so that the opening and the light emitting element face each other; an ultraviolet light transmitting window portion configured to close the opening; and a gasket positioned between the light source unit and the ultraviolet light transmitting window portion so as to prevent the object to be sterilized from entering a side with the light source unit in the accommodation space, wherein the gasket includes a reflector wall portion that has a light reflection property and that is recessed toward the light source unit so as to surround a side region of the light emitting element.

This application is a U.S. National Stage Application under 35 U.S.C § 371 of International Patent Application No. PCT/JP2023/007054 filed Feb. 27, 2023, which claims the benefit of priority under 35 U.S.C. § 119 to Japanese Patent Application No. 2022-031059 filed Mar. 1, 2022, the disclosures of all of which are hereby incorporated by reference in their entireties.

### TECHNICAL FIELD

The presently disclosed subject matter relates to a cap unit, a storage tank including the cap unit, and a flowing water sterilization device.

### BACKGROUND ART

For example, storage mechanisms for storing water (storage of water) that is used to make ice are provided in ice making machines or the like. Further, water circulation/transmission mechanisms that circulate and supply drinking water and industrial water are provided in water servers, drink servers, cooling water circulation devices (so-called chiller), and the like.

Since the storage tank and the water supply pipe in the storage mechanism and the transmission pipe in the water circulation/transmission mechanism are fixed in devices, they cannot be easily removed from the main body of the devices, and as a result the inside of each of the above-described portions (portions related to the storage and circulation of water such as a storage tank, a water supply pipe, and a transmission pipe) cannot be cleaned frequently. Therefore, there is a possibility of bacteria propagating in each of the above-mentioned portions.

Here, Patent Literatures 1 and 2 below disclose techniques for suppressing the propagation of bacteria inside a water supply pipe, a storage tank, or the like of an ice maker. Specifically, the invention disclosed in Patent Literature 1 includes an ultraviolet light irradiator that irradiates the water in the water supply pipe with ultraviolet light. Further, the invention disclosed in Patent Literature 2 includes an ultraviolet light irradiator that irradiates the water in the storage tank with ultraviolet light.

### CITATION LIST

### Patent Literature

Patent Literature 1: Japanese Patent Application Laid-Open No. 2020-20561
Patent Literature 2: Japanese Patent Application Laid-Open No. 2019-219095

### SUMMARY OF INVENTION

### Technical Problem

However, the ultraviolet light irradiators disclosed in Patent Literatures 1 and 2 do not include a portion for distributing ultraviolet light emitted from a light emitting element. As a result, the amount of ultraviolet light (illuminance) per unit area is reduced, resulting in a decrease in the sterilizing ability against bacteria. In addition, in a case where the water supply pipe or the storage tank is made of a resin, since the irradiation range of ultraviolet light cannot be controlled, there is a possibility that the water supply pipe or the storage tank is irradiated with ultraviolet light having a certain quantity of illuminance. As a result, degradation of the resin parts may be accelerated.

In view of the above-mentioned problems, one of the aspects of the presently disclosed subject matter is to provide a cap unit that is capable of enhancing the sterilizing ability with respect to an object to be sterilized while maintaining water stopping properties in a housing, and capable of irradiating a desired range with ultraviolet light, as well as a storage tank and a flowing water sterilization device.

### Solution to Problem

In order to solve the above-mentioned problems, a cap unit according to the presently disclosed subject matter includes:
a light source unit including a light emitting element and a substrate on which the light emitting element is mounted;
a housing that has an accommodation space for the light source unit and an opening at a predetermined position on a side that faces an object to be sterilized, the housing being configured to accommodate the light source unit so that the opening and the light emitting element face each other;
an ultraviolet light transmitting window portion configured to close the opening; and
a gasket positioned between the light source unit and the ultraviolet light transmitting window portion so as to prevent the object to be sterilized from entering a side with the light source unit in the accommodation space, wherein
the gasket includes a reflector wall portion that has a light reflection property and that is recessed toward the light source unit so as to surround a side region of the light emitting element.

According to this aspect of the presently disclosed subject matter, the gasket that is positioned between the light source unit and the ultraviolet light transmitting window portion to prevent an object to be sterilized from entering a side with the light source unit side includes the reflector wall portion having a light reflection property. Therefore, it is possible to control the irradiation range of the ultraviolet light from the light source unit to be a desired range (light distribution). As a result, the sterilizing ability with respect to the object to be sterilized can be enhanced while the cap unit can maintain the water stopping properties in the housing. Alternatively, even if resin parts are included in a cap unit attached body (for example, a storage tank or the like) to which the cap unit of the presently disclosed subject matter is attached, ultraviolet light distribution can be adjusted so as to avoid irradiation of the resin parts with ultraviolet light. Therefore, problems such as accelerated resin part degradation due to irradiation with ultraviolet light are suppressed.

In the cap unit according to the presently disclosed subject matter,
it is preferable that the gasket further include an outer extension portion configured to extend radially outward from the reflector wall portion and to abut against a predetermined position of the housing.

According to this aspect of the presently disclosed subject matter, the electronic components that are mounted on the substrate of the light source unit are covered by the reflector wall portion and the outer extension portion of the gasket. Accordingly, a space where ultraviolet light from the light emitting element can be blocked is ensured, and the electronic components can be accommodated in the space. As a result, relatively inexpensive electronic components having no ultraviolet resistance (resistance against UV) can be mounted on the substrate. In other words, according to this aspect of the presently disclosed subject matter, an excessive cost increase of the cap unit is not incurred.

In the cap unit according to the presently disclosed subject matter,
it is preferable that the reflector wall portion increase in diameter as it progresses from the light source unit toward the opening.

According to this aspect of the presently disclosed subject matter, the ultraviolet light component emitted from the light emitting element at a wide angle can also be distributed toward the opening side of the housing. As a result, the illuminance of the ultraviolet light irradiated within the predetermined range can be increased, and the sterilizing ability with respect to the object to be sterilized can be improved.

In the cap unit according to the presently disclosed subject matter,
it is preferable that the reflector wall portion extend along a height direction of the housing.

According to this aspect of the presently disclosed subject matter, the irradiation range of the ultraviolet light can be narrowed to be a desired range in contrast to the case where the reflector wall portion is not provided. As a result, the ultraviolet light distribution can be adjusted so that the ultraviolet light is prevented from being emitted to the resin parts of the cap unit attached body (for example, the storage tank or the like). Therefore, problems such as accelerated resin part degradation due to irradiation with ultraviolet light are suppressed.

In the cap unit according to the presently disclosed subject matter, it is preferable
that an electronic component different from the light emitting element be mounted on the substrate,
that a thick portion relatively close to the light emitting element and a thin portion relatively far from the light emitting element be formed in a diameter-increasing portion of the reflector wall portion, and
that the electronic component be opposed to a back surface region of the diameter-increasing portion of the reflector wall portion.

According to this aspect of the presently disclosed subject matter, by providing a thick portion that is relatively close to the light emitting element in the diameter-increasing portion of the reflector wall portion, it is possible to prevent the ultraviolet light from the light emitting element from being transmitted through the reflector wall portion. In addition, by providing a thin portion that is relatively far from the light emitting element in the diameter-increasing portion of the reflector wall portion, it is possible to expand the accommodation space for the electronic component.

Furthermore, in the cap unit according to the invention, it is preferable
that the reflector wall portion allow light from the light emitting element to enter the inside and to be diffused and reflected,
that an attachment made of a specular reflection material be provided on at least one of a front surface and a back surface of the diameter-increasing portion of the reflector wall portion.

According to this aspect of the presently disclosed subject matter, by providing an attachment made of a specular reflection material on at least one of the front surface or the back surface of the diameter-increasing portion of the reflector wall portion, it is possible to enhance the reflection characteristics with regards to the ultraviolet light from the light emitting element, or to prevent the ultraviolet light from the light emitting element from being transmitted through the reflector wall portion.

Further, a storage tank according to the presently disclosed subject matter includes:
a through hole provided at a predetermined position, and the above-mentioned cap unit configured to close the through hole, wherein
the storage tank is configured to store the object to be sterilized thereinside.

According to this aspect of the presently disclosed subject matter, the sterilizing ability with respect to the object to be sterilized can be enhanced while the storage tank can maintain the water stopping properties in the cap unit. Furthermore, ultraviolet light distribution can be adjusted so as to avoid irradiation of the resin parts of the storage tank with ultraviolet light. Therefore, problems such as accelerated resin part degradation due to irradiation with ultraviolet light are suppressed.

A flowing water sterilization device according to the presently disclosed subject matter can include:
a flow path pipe for passing the object to be sterilized,
a through hole provided at a predetermined position in the flow path pipe, and
the above-mentioned cap unit configured to close the through hole.

According to this aspect of the presently disclosed subject matter, the sterilizing ability with respect to the object to be sterilized can be enhanced while the flowing water sterilization device can maintain the water stopping properties in the cap unit. Furthermore, irradiation of the resin parts of the storage tank with ultraviolet light can be avoided. Therefore, problems such as accelerated resin part degradation due to irradiation with ultraviolet light are suppressed.

### Advantageous Effects of Invention

According to the presently disclosed subject matter, it is possible to provide a cap unit that is capable of enhancing the sterilizing ability with respect to an object to be sterilized while maintaining water stopping properties in a housing, and capable of irradiating a desired range with ultraviolet light, as well as a storage tank and a flowing water sterilization device.

### BRIEF DESCRIPTION OF DRAWINGS

FIG. 1 is a perspective view of a cap unit according to an embodiment of the presently disclosed subject matter.

FIG. 2 is a vertical cross-sectional view of the cap unit according to the present embodiment.

FIG. 3 is a vertical cross-sectional view of the cap unit according to the present embodiment.

FIG. 4 is a diagram showing a distribution of ultraviolet light illuminance irradiated from the cap unit according to the present embodiment.

FIG. 5 is an enlarged view of a gasket (reflector wall portion) of the present embodiment.

FIG. 6 is a diagram showing a modification of the cap unit according to the present embodiment.

FIG. 7 is a diagram showing another modification of the cap unit according to the present embodiment.

FIG. 8 is a diagram showing the cap unit of the present embodiment that is attached to a flowing water sterilization device.

FIG. 9 is a diagram showing the cap unit of the present embodiment attached to another flowing water sterilization device.

### DESCRIPTION OF EMBODIMENTS

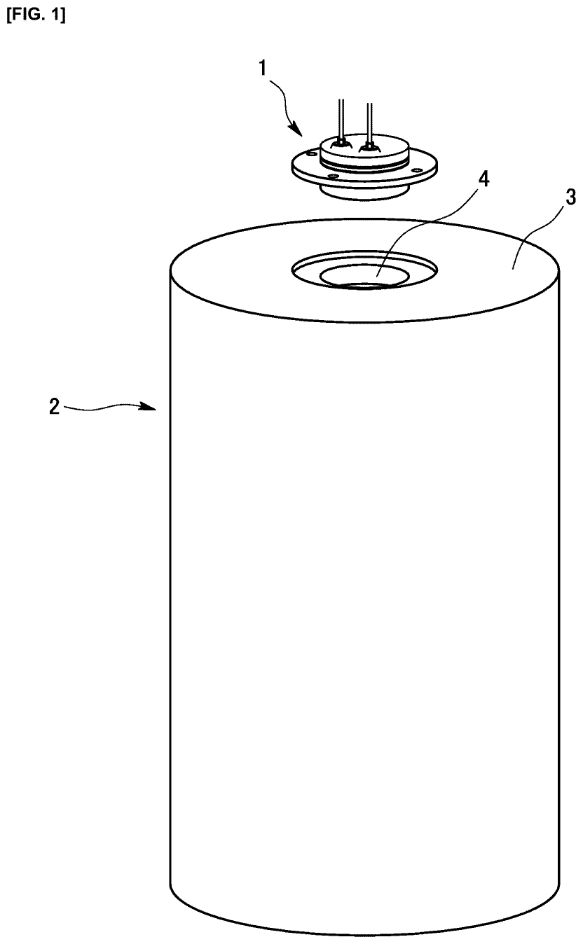

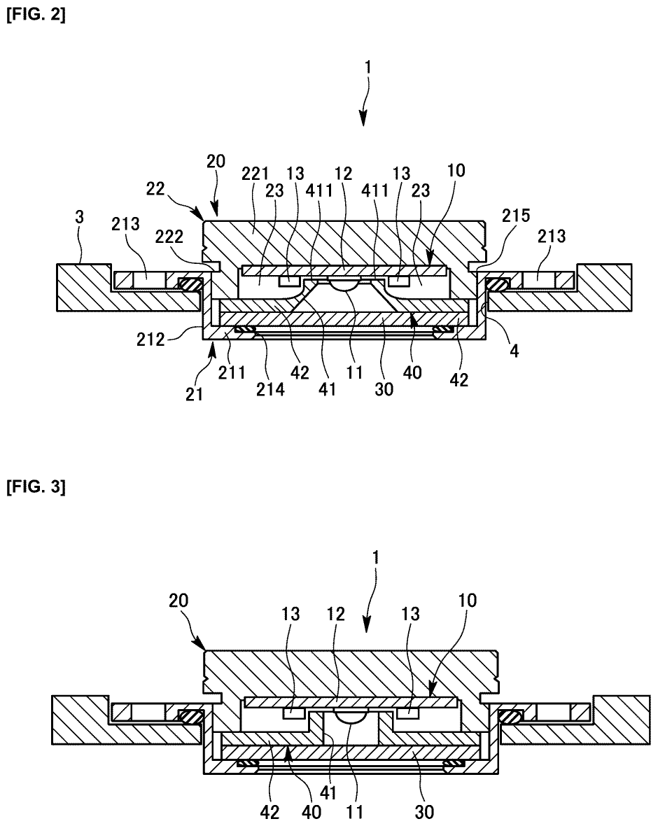

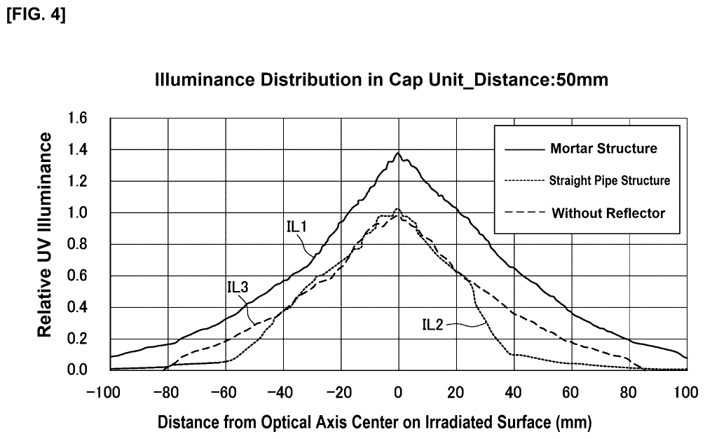

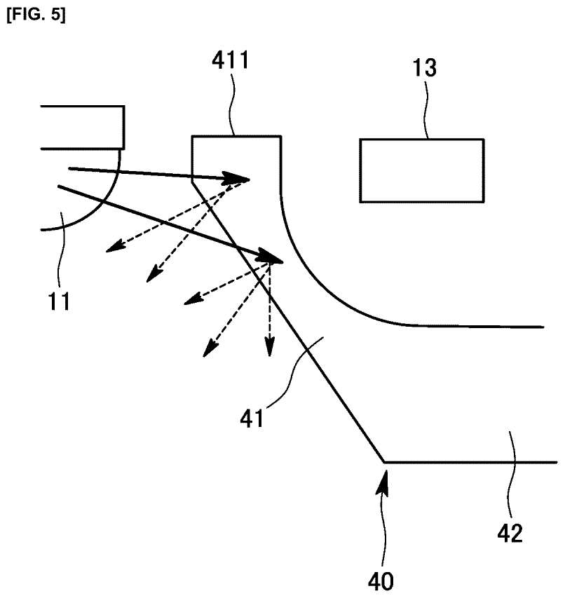

Hereinafter, a flowing water sterilization device according to an embodiment of the presently disclosed subject matter will be described in detail with reference to the drawings. First, a cap unit 1 according to the present embodiment will be described in detail with reference to FIGS. 1 to 5. Here, FIG. 1 is a perspective view of the cap unit 1. FIG. 2 and FIG. 3 are each a vertical cross-sectional view of the cap unit 1. FIG. 4 is a diagram showing a distribution of ultraviolet light illuminance emitted from the cap unit 1. FIG. 5 is an enlarged view of a gasket (reflector wall portion) provided in the cap unit 1.

As shown in FIG. 1, the cap unit 1 is attached to, for example, a storage tank 2 that stores an object to be sterilized (water or the like). More specifically, the cap unit 1 closes a through hole 4 provided in a predetermined place of the storage tank 2 (the place where the cap unit is to be attached may be the lower surface or the side surface of the storage tank 2 but is, in the present embodiment, the upper surface 3 of the storage tank 2). As will be described later, a light source unit (light emitting element) provided in the cap unit 1 is disposed so as to face the object to be sterilized in the storage tank 2. Thus, the ultraviolet light from the light emitting element of the cap unit 1 is emitted toward the object to be sterilized.

As shown in FIG. 2, the cap unit 1 according to the present embodiment includes a light source unit 10, a housing 20, an ultraviolet light transmitting window portion 30 (for example, quartz glass), a gasket 40, and the like.

The light source unit 10 includes the light emitting element 11 that emits ultraviolet light (for example, light having a peak wavelength within 100 nm to 400 nm), a substrate 12 on which the light emitting element 11 is mounted, and other electronic components 13 such as a power supply circuit and a connector that are also mounted on the substrate 12 to supply a current to the light emitting element 11. The type of the light emitting element 11 is not particularly limited, and examples thereof include an LED (Light emitting diode) and a semiconductor light emitting element such as a laser diode. The number of light emitting elements 11 of the present embodiment is one, but may be two or more.

Next, the housing 20 includes a housing main body 21 that fits into the through hole 4 of the storage tank 2. Here, the housing main body 21 includes a bottom wall 211 that faces the object to be sterilized in the storage tank 2, a side wall 212 that is connected to the edge of the bottom wall 211 and rises toward the top side of the housing main body 21, and a flange 213 that extends from the top end of the side wall 212 substantially horizontally and radially outward. As shown in FIG. 2, the flange 213 is in contact with the upper surface 3 of the storage tank 2 to support the housing main body 21.

In addition, a first opening 214 is formed in the bottom wall 211 of the housing main body 21 (at a predetermined position on the side facing the object to be sterilized). As shown in FIG. 2, the light emitting element 11 of the light source unit 10 faces the first opening 214. Further, the ultraviolet light transmitting window portion 30 closes the first opening 214. As a result, the ultraviolet light emitted from the light emitting element 11 passes through the first opening 214 (the ultraviolet light transmitting window portion 30) and is emitted onto the object to be sterilized in the storage tank 2.

The top surface of the housing main body 21 is open (a second opening 215). The housing 20 further includes a housing lid portion 22 that closes the second opening 215 of the housing main body 21. As shown in FIG. 2, the housing lid portion 22 includes a lid main body 221 and a lid side wall 222 extending from the lid main body 221 toward the bottom side.

As shown in FIG. 2, when the housing lid portion 22 closes the second opening 215 of the housing main body 21, the lid side wall 222 fits into the housing main body 21. As a result, an accommodation space 23 for the light source unit 10 is defined. Here, the light source unit 10 (the substrate 12) is attached to the inner wall of the lid main body 221.

Next, as shown in FIG. 2, the gasket 40 fills a gap formed between the ultraviolet light transmitting window portion 30 and the lid side wall 222 of the housing lid portion 22, thereby enhancing the water stopping properties of the housing 20. In addition, the gasket 40 includes a reflector wall portion 41 that has a light reflection property and that is recessed toward the light source unit 10 (the substrate 12) so as to surround the side region of the light emitting element 11. The reflector wall portion 41 opens at its front end 411 so that the light emitting element 11 faces the inside.

The gasket 40 further includes an outer extension portion 42 configured to extend radially outward from the reflector wall portion 41 and to abut against the inner wall of the housing 20 (the lid portion side wall 222 of the housing lid portion 22 in the present embodiment).

The gasket 40 of the present embodiment is made of polytetrafluoroethylene (PTFE). However, the material of the gasket 40 is not limited to this as long as it has light reflection properties. Other materials for the gasket 40 include a perfluoroethylene propene copolymer (FEP), perfluoroalkoxyalkane (PFA), and the like.

Here, the reflector wall portion 41 has a light distribution function with regards to ultraviolet light emitted from the light emitting element 11. However, the configuration of the reflector wall portion 41 is not limited as long as it can adjust the distribution of the ultraviolet light emitted from the light emitting element 11. Other examples of the reflector wall portion 41 include a mortar structure as shown in FIG. 2 (a configuration in which the diameter thereof increases progressively, from the light source unit 10 (the substrate 12) to the first opening 214), a straight pipe structure as shown in FIG. 3 (a configuration in which the reflector wall portion 41 extends along the height direction of the housing 20 (the vertical direction in the drawing)), and the like.

Further, as shown in FIG. 2, the electronic components 13 mounted on the substrate 12 of the light source unit 10 are covered by the reflector wall portion 41 and the outer extension portion 42 of the gasket 40. That is, according to the present embodiment, the electronic components 13 are provided with a space capable of blocking ultraviolet light from the light emitting element 11. This structure makes it possible to mount relatively inexpensive electronic components 13 having no ultraviolet resistance (resistance against UV) on the substrate 12. As a result, excessive cost increases of the cap unit are not incurred.

Here, at a position opposite to the bottom wall 211 side of the housing 20, a planar light receiving sensor was disposed substantially parallel to the width direction of the housing 20 (for example, a light receiving sensor was disposed at a position 50 mm away from the bottom wall 211), and three types of gaskets 40 were prepared, one with the reflector wall portion 41 having a mortar structure configuration (see FIG. 2), one with the reflector wall portion 41 having a straight pipe structure configuration (see FIG. 3), and one without the reflector wall portion 41. Then, illumination of ultraviolet light corresponding to the distance from the optical axis of the light emitting element 11 (the distance from an origin when the origin is defined as the intersection of the optical axis of the light emitting element 11 and the light receiving sensor) was measured. FIG. 4 shows the measurement results.

As shown in FIG. 4, the illuminance of ultraviolet light in the case where the reflector wall portion 41 had a mortar structure configuration was uniformly improved in the whole measurement range as compared with the case where the gasket was not provided with the reflector wall portion 41. In addition, the irradiation range of the ultraviolet light was narrowed more in the case where the reflector wall portion 41 has a straight pipe structure than that in the case where the gasket is not provided with the reflector wall portion 41 (that is, light distribution was able to be adjusted so as to narrow the irradiation range of the ultraviolet light).

As described above, when the reflector wall portion 41 has a mortar structure configuration, the illuminance of the ultraviolet light emitted from the light emitting element 11 can be increased, and the sterilizing ability with respect to the object to be sterilized can be improved. When the reflector wall portion 41 has a straight pipe structure configuration, the irradiation range of the ultraviolet light emitted from the light emitting element 11 can be narrowed to a desired range. As a result, for example, the ultraviolet light distribution can be adjusted so that ultraviolet light irradiation of the resin parts such as the storage tank 2 is prevented. Therefore, problems such as accelerated resin part degradation are suppressed.

When a resin (fluororesin or the like) such as PTFE is used as the gasket 40, as shown in FIG. 5, the ultraviolet light reaching the reflector wall portion 41 of the gasket 40 includes components that are diffusely reflected after entering the inside from the front surface of the reflector wall portion 41.

More specifically, the ultraviolet light emitted from the light emitting element 11 enters, in a substantially horizontally manner, the front end 411 of the reflector wall portion 41 at a location near the light emitting element 11. When a material that diffusely reflects light such as PTFE is used, the ultraviolet light passes through the vicinity of the front surface and then is reflected. Thus, if an appropriate thickness of the front end 411 of the reflector wall portion 41 is not ensured, there is a possibility that the ultraviolet light incident on the front end 411 side of the reflector wall portion 41 can pass through the reflector wall portion 41 and can be emitted toward the electronic components 13.

On the other hand, ultraviolet light emitted from the light emitting element 11 is obliquely incident on a portion away from the light emitting element 11. That is, the reflector wall portion 41 at a portion away from the light emitting element 11 has an increased apparent thickness with respect to the incident angle of the ultraviolet light. Therefore, the thickness of the reflector wall portion 41 at the portion away from the light emitting element 11 can be made thinner than the front end 411 of the reflector wall portion.

From this viewpoint, it is preferable to provide a space capable of appropriately accommodating the electronic components 13 mounted on the substrate 12 while the light blocking property toward the electronic components 13 is ensured. Specifically, as shown in FIG. 5, it is preferable to increase the thickness of the front end 411 of the reflector wall portion 41 and decrease the thickness of the reflector wall portion 41 from the thick front end to a predetermined position toward the outer extension portion 42. Due to such a change in the wall thickness of the reflector wall portion 41, in the present embodiment, the electronic components 13 can be disposed in a region overlapping with the inclined surface of the reflector wall portion 41 in the emission surface direction in the front view (the light emitting element 11 and the electronic components 13 other than the light emitting element 11 can be disposed so as to face each other via the front end 411 of the reflector wall portion 41).

Further, in a cross-sectional view as shown in FIG. 5, the back surface of the reflector wall portion 41 (the surface on the side facing the electronic components 13) has a curved surface shape recessed in a substantially arc shape toward the front surface of the reflector wall portion 41 (the surface on the side facing the light emitting element 11). By adopting such a configuration as the reflector wall portion 41, it is possible to expand the accommodation space for the electronic components 13 while the light blocking property of the ultraviolet light from the light emitting element 11 is ensured.

An embodiment of the presently disclosed subject matter has been described in detail above. However, the foregoing description is for the purpose of facilitating understanding of the present invention, and is not intended to limit the present invention. The present invention may include modifications and improvements without departing from the gist thereof. The present invention also includes equivalents thereof.

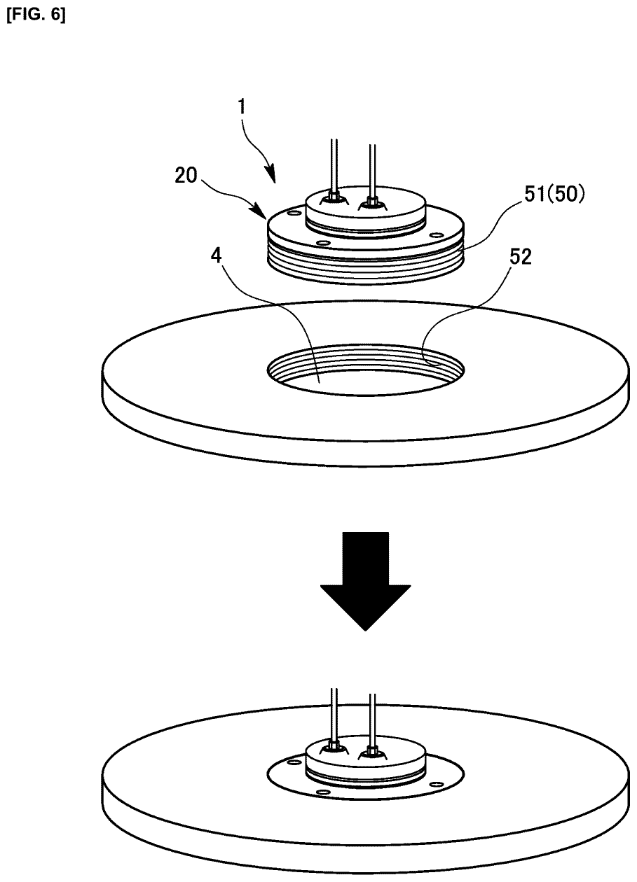

For example, as shown in FIG. 6, the cap unit 1 may further include a first attachment 50 having a male screw groove 51 formed radially outward on the side wall 212 of the housing main body 21. In this case, a female screw groove 52 to be engaged at the threads with the male screw groove 51 of the first attachment 50 is formed in the inner wall of the through hole 4 of the storage tank 2.

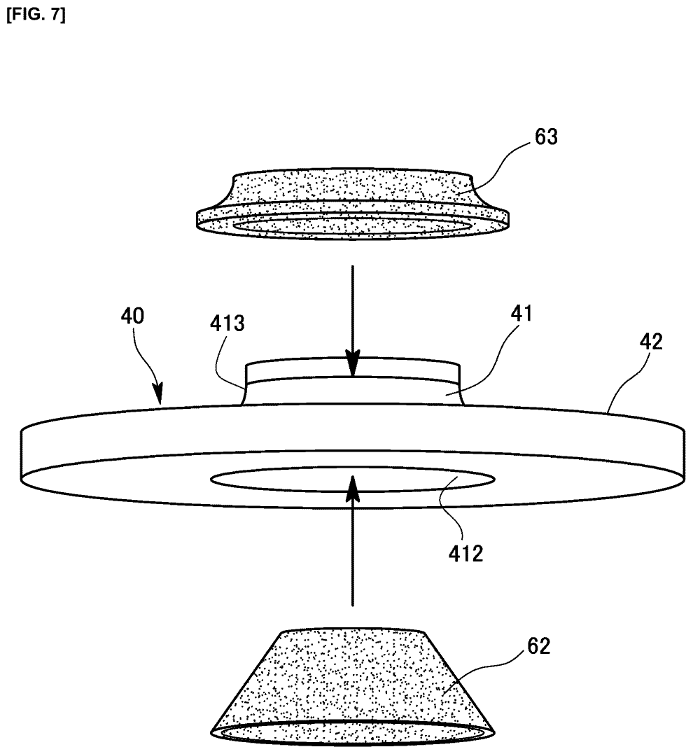

Further, as shown in FIG. 7, the cap unit 1 may include a second attachment 61 and a third attachment 62 made of a specular reflective material that cover the front surface 412 and the back surface 413 of the reflector wall portion 41 of the gasket 40. The specular reflective material is, for example, a metal such as aluminum. By attaching the second attachment 61, it is possible to improve the light distribution property with regards to the ultraviolet light emitted from the light emitting element 11 to the side of an object to be sterilized. Further, by attaching the third attachment 62, it is possible to prevent the ultraviolet light emitted from the light emitting element 11 from being transmitted through the reflector wall portion 41 and being emitted onto the side with the electronic components 13.

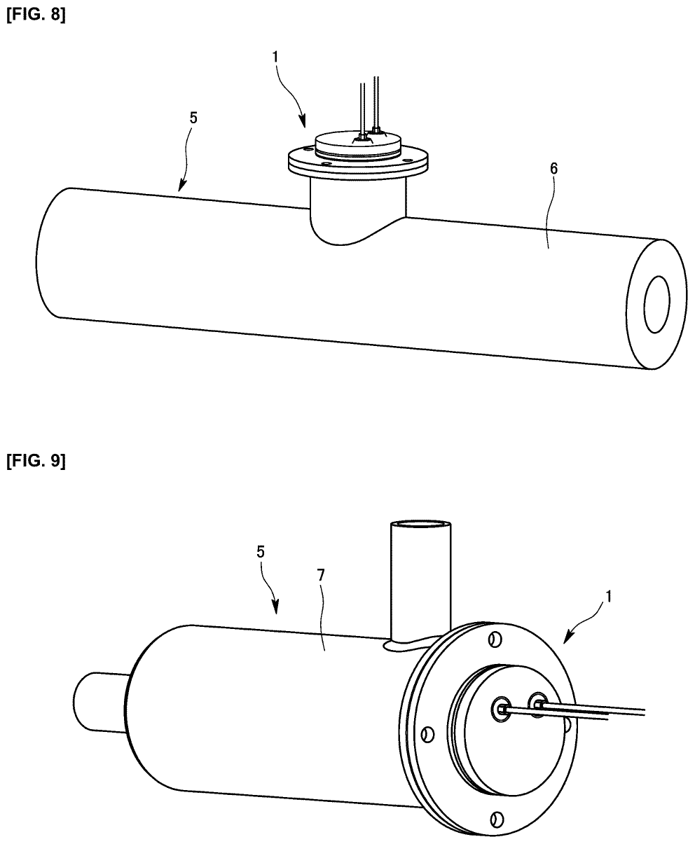

Further, the cap unit 1 according to the above-described embodiment is attached to the storage tank 2 that stores an object to be sterilized, but may be attached to, for example, a substantially I-shaped (see FIG. 8) or a substantially L-shaped flow path pipe 6, 7 (see FIG. 9) in the flowing water sterilization apparatus 5.

### INDUSTRIAL APPLICABILITY

The cap unit 1 according to the presently disclosed subject matter is attached to, for example, a storage tank of such as an icemaker, an ultraviolet light sterilization device, a water cleaner, a hot water supply device, a water supply pipe, a cooling water circulation device, a water server, a drink server, or the like. However, the use thereof is not limited to this.

### REFERENCE SIGNS LIST

1 . . . cap unit
10 . . . light source unit
   11 . . . light emitting element
   12 . . . substrate
   13 . . . electronic component
20 . . . housing
   21 . . . housing main body
      211 . . . bottom wall
      212 . . . side wall
      213 . . . flange
      214 . . . first opening
      215 . . . second opening
   22 . . . housing lid
      221 . . . lid main body
      222 . . . lid side wall
   23 . . . accommodation space for light source unit
30 . . . ultraviolet light transmitting window portion
40 . . . gasket
   41 . . . reflector wall portion
      411 . . . front end of reflector wall portion
      412 . . . front surface of reflector wall portion
      413 back surface of reflector wall portion
   42 . . . outer extension portion
50 . . . first attachment
   51 . . . male screw groove
   52 . . . female screw groove
61 . . . second attachment
62 . . . third attachment
2 . . . storage tank
3 . . . upper surface of storage tank
4 . . . through hole provided in storage tank
5 . . . flowing water sterilization apparatus
6 . . . substantially I-shaped flow path pipe
7 . . . substantially L-shaped flow path pipe

1. A cap unit comprising:
a light source unit including a light emitting element and a substrate on which the light emitting element is mounted;
a housing that has an accommodation space for the light source unit and an opening at a predetermined position on a side that faces an object to be sterilized, the housing being configured to accommodate the light source unit so that the opening and the light emitting element face each other;
an ultraviolet light transmitting window portion configured to close the opening; and
a gasket positioned between the light source unit and the ultraviolet light transmitting window portion so as to prevent the object to be sterilized from entering a side with the light source unit in the accommodating space, wherein
the gasket includes a reflector wall portion that has a light reflection property and that is recessed toward the light source unit so as to surround a side region of the light emitting element.

2. The cap unit according to claim 1, wherein the gasket further includes an outer extension portion configured to extend radially outward from the reflector wall portion and to abut against the housing.

3. The cap unit according to claim 2, wherein:
light emitted from the light emitting element is directly incident on the reflector wall portion; and
the reflector wall portion increases in diameter as it progresses from the light source unit toward the opening.

4. The cap unit according to claim 2, wherein the
reflector wall portion extends along a height direction of the housing.

5. The cap unit according to claim 3, wherein:
an electronic component different from the light emitting element is mounted on the substrate;
a thick portion relatively close to the light emitting element and a thin portion relatively far from the light emitting element are formed in a diameter-increasing portion of the reflector wall portion; and
the electronic component is opposed to a back surface region of the diameter-increasing portion of the reflector wall portion.

6. The cap unit according to claim 3, wherein:
the reflector wall portion allows light from the light emitting element to enter the inside and to be diffused and reflected; and
an attachment made of a specular reflection material is provided on at least one of a front surface and a back surface of the diameter-increasing portion of the reflector wall portion.

7. A storage tank comprising:
a through hole, and
the cap unit according to claim 1, the cap unit being configured to close the through hole, wherein
the storage tank is configured to store the object to be sterilized thereinside.

8. A flowing water sterilization device comprising:
a flow path pipe for passing the object to be sterilized;
a through hole provided in the flow path pipe, and
the cap unit according to claim 1, the cap unit being configured to close the through hole.

9. The cap unit according to claim 2, wherein the gasket is made of any of polytetrafluoroethylene (PTFE), a perfluoroethylene propene copolymer (FEP), and perfluoroalkoxyalkane (PFA).

10. The cap unit according to claim 2, wherein:
the housing includes a housing lid portion and a housing main body;
the housing main body has a bottom wall where the opening is formed, a side wall that is connected to an edge of the bottom wall and rises toward a top side, and a second opening in a top surface;
the housing lid portion has a lid main body, and a lid side wall extending from the lid main body toward a bottom side;
the lid side wall fits into the second opening to define the accommodation space;
the gasket fills a gap formed between the ultraviolet light transmitting window portion and a bottom surface of the lid side wall to prevent the object to be sterilized from entering.

11. The cap unit according to claim 2, wherein the housing has a flange that extends substantially horizontally and radially outward.

12. The cap unit according to claim 2, further comprising an attachment having a screw groove formed radially outward on the housing.

13. The cap unit according to claim 1, wherein the gasket is made of any of polytetrafluoroethylene (PTFE), a perfluoroethylene propene copolymer (FEP), and perfluoroalkoxyalkane (PFA).

14. The cap unit according to claim 1, wherein:
the housing includes a housing lid portion and a housing main body;
the housing main body has a bottom wall where the opening is formed, a side wall that is connected to an edge of the bottom wall and rises toward a top side, and a second opening in a top surface;
the housing lid portion has a lid main body, and a lid side wall extending from the lid main body toward a bottom side;
the lid side wall fits into the second opening to define the accommodation space;
the gasket fills a gap formed between the ultraviolet light transmitting window portion and a bottom surface of the lid side wall to prevent the object to be sterilized from entering.

15. The cap unit according to claim 1, wherein the housing has a flange that extends substantially horizontally and radially outward.

16. The cap unit according to claim 1, further comprising an attachment having a screw groove formed radially outward on the housing.

17. The cap unit according to claim 1, wherein:
light emitted from the light emitting element is directly incident on the reflector wall portion; and
the reflector wall portion increases in diameter as it progresses from the light source unit toward the opening.

18. The cap unit according to claim 1, wherein the reflector wall portion extends along a height direction of the housing.

19. The cap unit according to claim 5, wherein:
the reflector wall portion allows light from the light emitting element to enter the inside and to be diffused and reflected; and
an attachment made of a specular reflection material is provided on at least one of a front surface and a back surface of the diameter-increasing portion of the reflector wall portion.
